(19) United States
(12) Patent Application Publication
(10) Pub. No.: US 2024/0382741 A1
(43) Pub. Date: Nov. 21, 2024

(54) RENAL PUMP SUPPORT SYSTEM

(71) Applicant: Boston Scientific Scimed, Inc., Maple Grove, MN (US)

(72) Inventor: Corey N. Meyer, Zimmerman, MN (US)

(73) Assignee: Boston Scientific Scimed, Inc., Maple Grove, MN (US)

(21) Appl. No.: 18/666,077

(22) Filed: May 16, 2024

Related U.S. Application Data
(60) Provisional application No. 63/467,182, filed on May 17, 2023.

(51) Int. Cl. A61M 60/30; A61M 60/13; A61M 60/226; A61M 60/414; A61M 60/546

(52) U.S. Cl. CPC A61M 60/30; A61M 60/13; A61M 60/226; A61M 60/414; A61M 60/546

(57) ABSTRACT

An example renal support pump includes a renal support device including an outer catheter shaft having a longitudinal axis and a distal end region coupled to a renal pump assembly, wherein the renal pump assembly includes a frame and an impeller assembly, wherein the impeller assembly is disposed within the frame, and wherein the impeller assembly is configured to rotate relative to the frame about the longitudinal axis of the catheter shaft.

### CROSS-REFERENCE TO RELATED APPLICATIONS

This application claims the benefit of priority under 35 U.S.C. § 119 of U.S. Provisional Application No. 63/467,182, filed May 17, 2023, the entire disclosure of which is hereby incorporated by reference.

### TECHNICAL FIELD

The present disclosure relates to renal support device systems. More specifically, the disclosure relates to a renal support pump capable of being advanced over a circulatory support device.

### BACKGROUND

Percutaneous circulatory support devices such as blood pumps can provide transient cardiac support in patients whose heart function or cardiac output is compromised. Such devices may be delivered percutaneously from the femoral artery, retrograde through the descending aorta, over the aortic arch, through the ascending aorta across the aortic valve, and into the left ventricle, whereby the circulatory support device (e.g., the blood pump) may pump blood from the left ventricle to the rest of the body.

However, in some instances, a patient whose heart function or cardiac output is compromised may experience renal dysfunction. While utilization of a blood pump may improve overall cardiac output, renal function can decline post-implantation of a circulatory support device. Accordingly, there is an ongoing need to provide a renal support pump capable of being used during a cardiac procedure to support the kidneys in conjunction with a circulatory support device used to support the heart. Renal support pumps capable of being advanced over a circulatory support device and used in conjunction with the cardiac support device during a cardiac procedure are disclosed herein.

### SUMMARY

This disclosure provides design, material, manufacturing method, and use alternatives for medical devices and/or systems. An example renal support pump includes a renal support device including an outer catheter shaft having a longitudinal axis and a distal end region coupled to a renal pump assembly, wherein the renal pump assembly includes a frame and an impeller assembly, wherein the impeller assembly is disposed within the frame, and wherein the impeller assembly is configured to rotate relative to the frame about the longitudinal axis of the catheter shaft.

Alternatively or additionally to any of the embodiments above, the impeller assembly includes a first impeller blade coupled to an impeller shaft.

Alternatively or additionally to any of the embodiments above, the impeller shaft includes a first end region and a second end region, and wherein both the first end region and the second end region of the impeller shaft are coupled to an electrical drive cable.

Alternatively or additionally to any of the embodiments above, the impeller shaft includes a lumen extending therein, and wherein the electrical drive cable is configured to extend within the lumen of the impeller shaft.

Alternatively or additionally to any of the embodiments above, a proximal end of the electrical drive cable is coupled to a motor core.

Alternatively or additionally to any of the embodiments above, the electrical drive cable and the motor core are configured to rotate around the inner shaft.

Alternatively or additionally to any of the embodiments above, the first blade extends from the first end region of the impeller shaft to a second end region of the impeller shaft.

Alternatively or additionally to any of the embodiments above, further comprising a motor assembly including a motor core disposed within a motor housing, wherein the motor housing is coupled to a distal end region of the outer catheter shaft.

Alternatively or additionally to any of the embodiments above, the electrical drive cable extends along an inner surface of the outer catheter.

Alternatively or additionally to any of the embodiments above, the electrical drive cable includes a lumen and wherein the inner catheter shaft is configured to extend within the lumen of the electrical drive cable.

Alternatively or additionally to any of the embodiments above, the electrical drive cable extends circumferentially around an outer surface of the inner catheter shaft.

Alternatively or additionally to any of the embodiments above, the motor core includes an aperture, and wherein the inner catheter shaft is configured to extend through the aperture of the motor core.

Alternatively or additionally to any of the embodiments above, further comprising a console coupled to the motor assembly.

Alternatively or additionally to any of the embodiments above, the rotation of the impeller assembly is configured to create a high-pressure region within a body vessel of a patient.

An example renal pump system includes a console including a processor, a motor assembly coupled to the console and an outer catheter having a first end coupled to the motor assembly and a second end coupled to a renal pump. Further, the renal pump includes a frame and an impeller assembly. Further, the impeller assembly is configured to rotate relative to the frame, and wherein rotation of the impeller assembly is configured to create a high-pressure region within a body vessel of a patient.

Alternatively or additionally to any of the embodiments above, the impeller assembly includes a first impeller blade coupled to an impeller shaft.

Alternatively or additionally to any of the embodiments above, the impeller shaft includes a first end region and a second end region, and wherein both the first end region and the second end region of the impeller shaft are coupled to an electrical drive cable.

Alternatively or additionally to any of the embodiments above, the impeller shaft includes a lumen extending therein, and wherein the electrical drive cable is configured to extend within the lumen of the impeller shaft.

Alternatively or additionally to any of the embodiments above, the first impeller blade extends from the first end region of the impeller shaft to a second end region of the impeller shaft.

An example method for positioning a renal pump system adjacent the renal arteries of a patient includes advancing a renal pump device adjacent to a renal artery of the patient. The renal pump device includes an outer catheter shaft having a longitudinal axis and a distal end region coupled to the renal pump device, wherein the renal pump device includes a frame and an impeller assembly, wherein the impeller assembly is disposed within the frame, and wherein the impeller assembly is configured to rotate relative to the frame about the longitudinal axis of the catheter shaft.

Alternatively or additionally to any of the embodiments above, wherein rotation of the impeller assembly is configured to create a high-pressure region adjacent the renal arteries of the patient.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

FIG. 1 depicts a renal support system and a percutaneous circulatory support system, including a renal support device positioned adjacent the renal arteries and a circulatory support device positioned relative to the heart of a patient;

FIG. 2 is a schematic block diagram of a console management system;

FIG. 3 depicts a portion of the circulatory support system shown in FIG. 1 positioned in the heart of a patient;

FIG. 4 depicts a cross-section of a portion of the renal support system shown in FIG. 1;

FIG. 5 depicts a portion of the renal support system shown in FIG. 1 positioned adjacent the renal arteries of a patient;

FIG. 6 depicts a cross-section of the example renal support device shown in FIG. 5;

FIG. 7 depicts an alternative embodiment of the renal support system shown in FIG. 1 positioned adjacent the renal arteries of a patient;

FIG. 8 depicts an alternative embodiment of the renal support system shown in FIG. 1 positioned adjacent the renal arteries of a patient.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

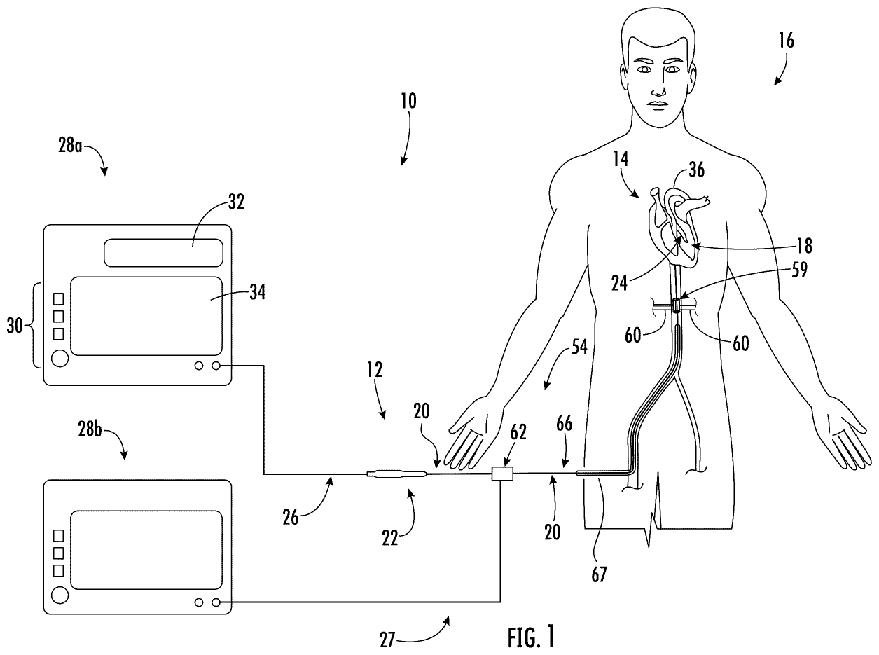

FIG. 1 illustrates an example percutaneous circulatory system 10 including a circulatory support device 12 positioned in the heart 14 of a patient 16. The circulatory support device 12 may include a flexible elongated catheter shaft 20 having a first end attached to a handle 22 and a second end attached to a blood pump 24. FIG. 1 illustrates the blood pump 24 positioned in the left ventricle 18 of the patient 16. The blood pump 24 may be delivered (e.g., tracked) to the ventricle 18 percutaneously over a guidewire. For example, the catheter shaft 20 and blood pump 24 may be tracked over a guidewire through the femoral artery, past the renal arteries 60 and the descending aorta, over the aortic arch, through the ascending aorta 37, past the aortic valve and into the left ventricle 18.

FIG. 1 further illustrates that the handle 22 may include a distal end region attached to the catheter shaft 20 and a proximal end region attached to an electrical power cable 26. The electrical power cable 26 may include a distal end region connected to a console 28a. It can be appreciated that the handle 22 may include one or more actuators (e.g., buttons, levers, dials, switches, etc.) designed to permit a clinician to control various functions of the blood pump 24. For example, a clinician may be able to control the speed of the motor and/or an impeller located in the blood pump 24 via actuation of one or more actuators located on the handle 22.

Additionally, FIG. 1 illustrates that the console 28a may include one or more control knobs (e.g., buttons, knobs, dials, etc.) 30 and/or one or more displays. For example, FIG. 1 illustrates the console 28a may include a first display 32 and a second display 34. It can be appreciated that the console 28a may include more than two displays. Additionally, while FIG. 1 illustrates the first display 32 and the second display 34 integrated into the console 28a, it is contemplated that the circulatory system 10 may be designed such that the first display 32, the second display 34 or both the first display 32 and the second display 34 are separate, distinct components of the circulatory system 10. In other words, the first display 32, the second display 34 or both the first display 32 and the second display 34 may be separate stand-alone displays, apart from the console 28a. In some examples, the first display 32 and the second display 34 may get their respective data from separate sources.

In some examples, the second display 34 may be designed to attach to the console 28a and/or the first display 32. For example, the first display 32 may be integrated into the console 28a while the second display 34 may be configured to attach to portion of the console 28a. In yet other examples, both the first display 32 and the second display 34 may be a separate stand-alone display whereby the second display 34 may be configured to attach to the first display 32, or wherein the first display 32 may be configured to attach to the second display 34.

FIG. 1 further illustrates an example renal support device 54 including a renal support pump 59 positioned adjacent the renal arteries 60 of the patient 16. The renal support device 54 may include a flexible elongated catheter shaft 66 having a first end attached to a motor assembly 62 and a second end attached to the renal support pump 59. As will be discussed in greater detail herein, the renal support pump 59 may be delivered (e.g., tracked) to a position adjacent the renal arteries 60 over the flexible elongated catheter shaft 20 of the circulatory support device 12. Further, FIG. 1 illustrates that the motor assembly 62 may be attached to a first end of an electrical power cable 27. The electrical power cable 27 may further include a second end connected to a console 28b. It can be appreciated that the console 28b may include one or more actuators (e.g., buttons, levers, dials, switches, etc.) designed to permit a clinician to control various functions of the renal support pump 59.

FIG. 1 further illustrates that the renal support device 54 may further include a delivery catheter 67 positioned radially outward of the outer shaft 66. The delivery catheter 67 may be configured to move relative to the outer shaft 66 and the renal support pump 59. For example, the outer shaft 67 may be configured to be advanced over the renal support pump 59, whereby the renal support pump 59 may collapse such that it can be positioned within the lumen of the delivery catheter 67 in a delivery configuration. Retraction of the delivery catheter 67 may permit the renal support pump to expand into a deployed configuration after being tracked to a target deployment site (e.g., the renal arteries).

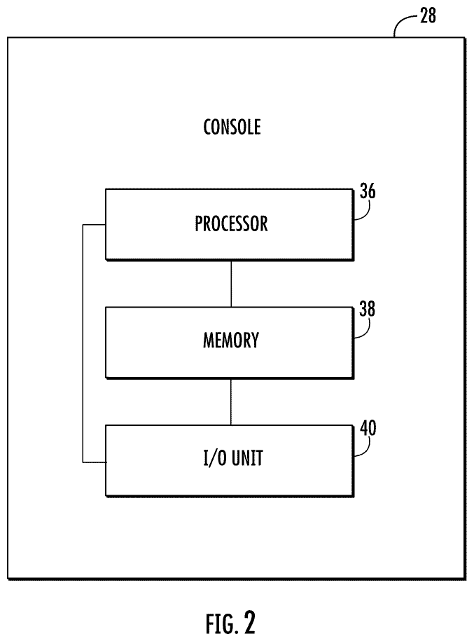

FIG. 2 illustrates that each of the console 28a and the console 28b may include, among other suitable components, one or more processors 36, memory 38, and an I/O unit 40. The processors 36 of the consoles 28a, 28b may include a single processor or more than one processor working individually or with one another. The processor 36 may be configured to execute instructions, including instructions that may be loaded into the memory 38 and/or other suitable memory. Example processor components may include, but are not limited to, microprocessors, microcontrollers, multi-core processors, graphical processing units, digital signal processors, application specific integrated circuits (ASICs), field programmable gate arrays (FPGAs), discrete circuitry, and/or other suitable types of data processing devices. In some examples, the processor 36 of the consoles 28 may be configured to execute program instructions. Program instructions may include, for example, firmware, microcode or application code that is executed by the processor 36, a microprocessor and/or microcontroller. The one or more processors 36 may be configured to each manage different functions. They may also be configured to concurrently perform the same functions (e.g., redundant system). Further yet, they may be configured such that a first processor 36 performs a given function and second processor 36 checks the result of the function of the first processor 36 for correctness (e.g., command-monitor system).

The memory 38 of the consoles 28a, 28b may include a single memory component or more than one memory component each working individually or with one another. Example types of memory may include random access memory (RAM), EEPROM, FLASH, suitable volatile storage devices, suitable non-volatile storage devices, persistent memory (e.g., read only memory (ROM), hard drive, Flash memory, optical disc memory, and/or other suitable persistent memory) and/or other suitable types of memory. The memory 38 may be or may include a non-transitory computer readable medium.

The I/O units 40 of the consoles 28a, 28b may include a single I/O component or more than one I/O component each working individually or with one another. Example I/O units 40 may be any type of communication port configured to communicate with other components of the circulatory system 10. Example types of I/O units 45 may include wired ports, wireless ports, radio frequency (RF) ports, Low-Energy Bluetooth ports, Bluetooth ports, Near-Field Communication (NFC) ports, HDMI ports, Wi-Fi ports, Ethernet ports, VGA ports, serial ports, parallel ports, component video ports, S-video ports, composite audio/video ports, DVI ports, USB ports, optical ports, and/or other suitable ports.

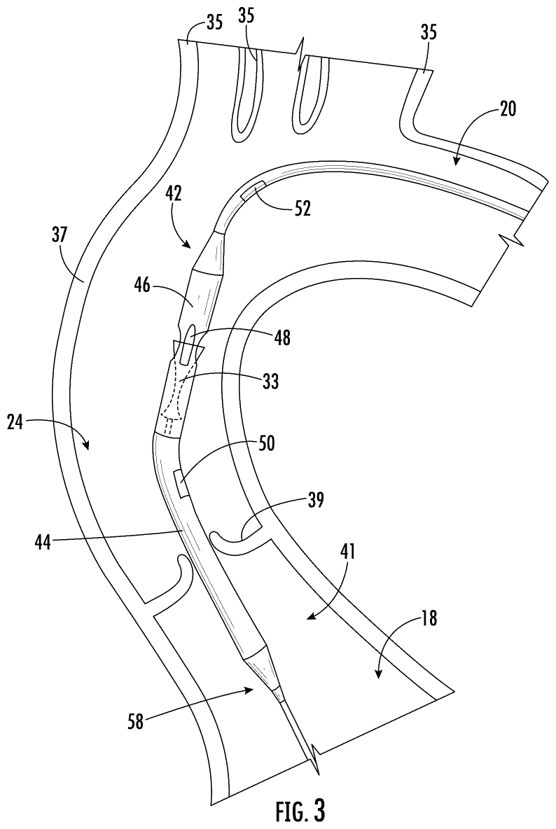

FIG. 3 illustrates the blood pump 24 of the percutaneous circulatory system 10 extending from the ascending aorta 37 to the left ventricle 18 of a patient 16. The blood pump 24 may include a cannula 44 having a proximal end attached to a distal end of an impeller housing 46. A proximal end 42 of the impeller housing 46 may be attached to a distal end of the catheter shaft 20. FIG. 3 illustrates that, in some examples, the blood pump 24 may be positioned within the heart 14 such that the cannula 44 passes through the aortic valve 39, whereby a distal end region 41 of the cannula 44 may be positioned within the left ventricle 18. As discussed herein, the blood pump 24 may be tracked over a guidewire to its position illustrated in FIG. 3.

FIG. 3 further illustrates that the shaft 20 of the circulatory support device 12 may include one or more blood inlets 58 located on a distal end region 41 of the cannula 44, and one or more blood outlets 48 positioned along the impeller housing 46. In some examples, the blood pump 24 may be positioned within the heart 14 such that the one or more blood inlets 58 positioned along the distal end region 41 of the cannula 44 may be positioned in the left ventricle 18 and the one or more blood outlets 48 located along the impeller housing 46 may be positioned in the ascending aorta 37.

Additionally, the blood pump 24 may include an electrically powered motor that drives rotation of the impeller 33 which may be positioned within the impeller housing 46. In some examples, the motor may power the rotation of the impeller 33 via electromagnetic induction. The spinning impeller 33 may draw blood from the left ventricle 18 (via the one or more blood inlets 58 located on a distal region of the cannula 44) into the ascending aorta 37 (via the one or more blood outlets 48 located along the impeller housing 46). In other words, an electrically powered motor drives the impeller 33 to pump blood from the left ventricle 18 through the aortic valve 39 and into the ascending aorta 37.

Additionally, the circulatory support device 12 may include one or more sensors coupled to the cannula 44, the impeller housing 46 and/or the catheter shaft 20. The one or more sensors coupled to the cannula 44, the impeller housing 46 and/or the catheter shaft 20 may be designed to monitor blood pressures (e.g., arterial pressure, venous pressure), blood velocity, or other relevant cardiac parameters. Additionally, the one or more sensors of the circulatory support device 12 coupled to the cannula 44, the impeller housing 46 and/or the catheter shaft 20 may be designed to monitor other parameters of the circulatory system 10, the circulatory support device 12 and/or the patient 16.

FIG. 3 illustrates that, in some examples, the percutaneous circulatory system 10 may include a sensor 50 (e.g., flow sensor, position sensor, etc.) positioned along a distal end region of the cannula 44. For example, in which the sensor 50 is a flow sensor, the flow sensor 50 may be designed to sense the flowrate of blood (e.g., blood velocity) adjacent to the flow sensor 50. For example, the flow sensor 50 may be designed to directly measure the velocity of blood passing from the left ventricle 18, through the aortic valve 39 and into the ascending aorta 37. Any of the example flow sensors described herein may include an optical sensor, ultrasound sensor, electromagnetic sensor, thermoconvection sensor, etc. Additionally, for examples in which the sensor 50 is a position sensor, the sensor 50 may be able to determine the positioned of the blood pump 24 relative to the left ventricle 18 and/or the aorta 37 based on a sensed measurement of the blood velocity passing adjacent to the sensor 50.

FIG. 3 further illustrates that, in some examples, the percutaneous circulatory system 10 may include a flow sensor 52 positioned along a distal end region of the catheter shaft 20. In some examples, the flow sensor 52 may be positioned superior to the subclavian arteries 35. In other examples, the flow sensor 52 may be positioned inferior to the subclavian arteries 35. In yet other examples, the flow sensor 52 may be positioned about 1 cm to 10 cm from the proximal end of the impeller housing 46, or about 2 cm to 9 cm from the proximal end of the impeller housing 46, about 3 cm to 7 cm from the proximal end of the impeller housing 46, or about 4 cm to 6 cm from the proximal end of the impeller housing 46, or about 5 cm from the proximal end of the impeller housing 46. The flow sensor 52 may be designed to sense the flowrate of blood (e.g., blood velocity) adjacent to the flow sensor 52. For example, the flow sensor 52 may be designed to directly measure the velocity of blood passing from the left ventricle 18, through the aortic valve 39 and into the ascending aorta 37. Any of the example flow sensors described herein may include an optical sensor, ultrasound sensor, electromagnetic sensor, thermoconvection sensor, etc.

Additionally, it can be appreciated that, in some examples, the flow sensor 52 may be positioned within the descending aorta, superior to the celiac trunk, inferior to the celiac trunk, superior to the renal arteries and/or inferior to renal arteries.

In some instances, the position of the blood pump 24 (including the position of the blood inlets 58, the blood outlets 48 and/or the impeller 33) may be determined by comparing a blood velocity measurement taken at the flow sensor 50 with a blood velocity measurement taken at the flow sensor 52. For example, the processing components 36 of the percutaneous circulatory system 10 may include an algorithm designed to receive and compare the blood velocity data sent from the flow sensor 50 with the blood velocity data from the flow sensor 52. It can be appreciated that the velocity of blood passing by the flow sensor 50 may be less than the velocity of blood passing by the flow sensor 52 because the velocity of blood passing by the flow sensor 52 may be increased (e.g., assisted) by the impeller 33. In other words, the velocity of blood exiting the blood outlets 48 may be greater than the blood passing by the flow sensor 50 and the blood entering the blood inlets 58. Further, knowing the distance between the flow sensor 50 and the flow sensor 52, the processing components 36 may be able to calculate the relative position of the impeller housing (positioned between the flow sensor 50 and the flow sensor 52) by comparing the blood velocity measurement taken at the flow sensor 50 with the blood velocity measurement taken at the flow sensor 52.

Additionally, it can be appreciated that, for any of the flow sensors described herein (e.g., flow sensor 50, flow sensor 52, etc.), the blood velocity directly measured by a given flow sensor (e.g., flow sensor 50, flow sensor 52, etc.) may be utilized to calculate a measurement of cardiac output. Cardiac output may be defined as the amount of blood pumped per unit of time (e.g., the volumetric flowrate of blood within the body). Cardiac output may be calculated by multiplying the stroke volume (e.g., the volume of blood exiting the left ventricle per stroke) times the heart rate (e.g., the number of strokes of the left ventricle per unit time).

It can be further appreciated that, in some examples, the processing components 36 of the percutaneous circulatory system 10 may include an algorithm designed to receive and compare the blood velocity data sent from any of the flow sensors described herein (e.g., flow sensor 50, flow sensor 52, etc.) and use it to calculate the cardiac output of the heart 14. Further, any of the flow sensors described herein (e.g., the flow sensor 50 and the flow sensor 52) may be coupled to and/or incorporate an impedance sensor, whereby the impedance sensor may be utilized to determine (e.g., calculate) the diameter of a body vessel (e.g., the ascending aorta, descending aorta, etc.) adjacent to a flow sensor (e.g., the flow sensor 50 and the flow sensor 52). It can be appreciated that the diameter of a body vessel (e.g., the ascending aorta, descending aorta, etc.) may be utilized by the processing components 36 to calculate the cardiac output of the heart 14.

Further yet, one or more components of the percutaneous circulatory system 10 may be coupled to an ultrasound system capable of utilizing ultrasound to determine the diameter of a body vessel (e.g., the ascending aorta, descending aorta, etc.) adjacent to a flow sensor (e.g., the flow sensor 50 and the flow sensor 52). The ultrasound system may communicate with the processing components 36 of the percutaneous circulatory system 10. Accordingly, the processing components 36 of the percutaneous circulatory system 10 may include an algorithm capable of receiving data from the ultrasound system corresponding to the diameter of a body vessel (e.g., the ascending aorta, descending aorta, etc.) adjacent to a flow sensor (e.g., the flow sensor 50 and the flow sensor 52). Further, the data received from the ultrasound system may be utilized by the processing components 36 of the percutaneous circulatory system 10 to calculate the cardiac output of the heart 14.

In yet another example, the percutaneous circulatory system 10 may include a conductance catheter configured to measure the conductance of blood between two equally spaced electrodes positioned on the conductance catheter, whereby the volume of blood may be calculated based on the conductance reading. Further, knowing the volume of blood in a blood vessel at an instance in time may be used to estimate the diameter of the vessel adjacent to the electrodes. Accordingly, the processing components 36 of the percutaneous circulatory system 10 may include an algorithm capable of receiving data from the conductance catheter corresponding to the diameter of a body vessel (e.g., the ascending aorta, descending aorta, etc.). Further, the data received from the conductance catheter may be utilized by the processing components 36 of the percutaneous circulatory system 10 to calculate the cardiac output of the heart 14.

It can be appreciated that any of the flow sensors described herein (e.g., flow sensor 50, flow sensor 52, etc.) may send signals to the console 28 and/or the processing components 26 via a wireless connection (e.g., a Bluetooth connection). In other examples, any of the flow sensors described herein (e.g., flow sensor 50, flow sensor 52, etc.) may be hardwired to the console 28 and/or the processing components 26.

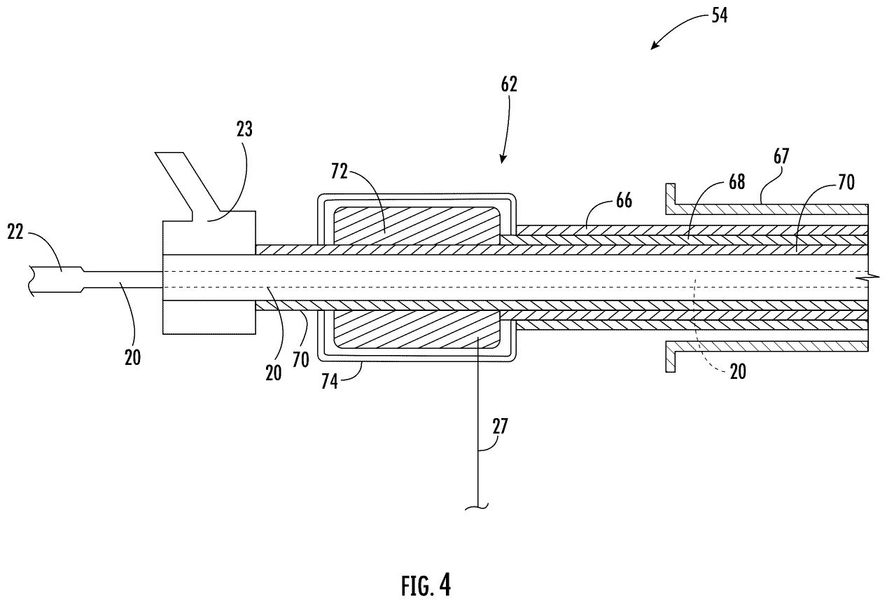

FIG. 4 illustrates a cross-section of a portion of the renal support device 54 including the motor assembly 62. As shown in FIG. 4, the motor assembly 62 may include a motor core 72. As discussed herein, the motor core 72 may be coupled to a power source in the console 28b via an electrical power cable 27. Additionally, the motor assembly 62 may include a motor housing 74 surrounding the motor core 72.

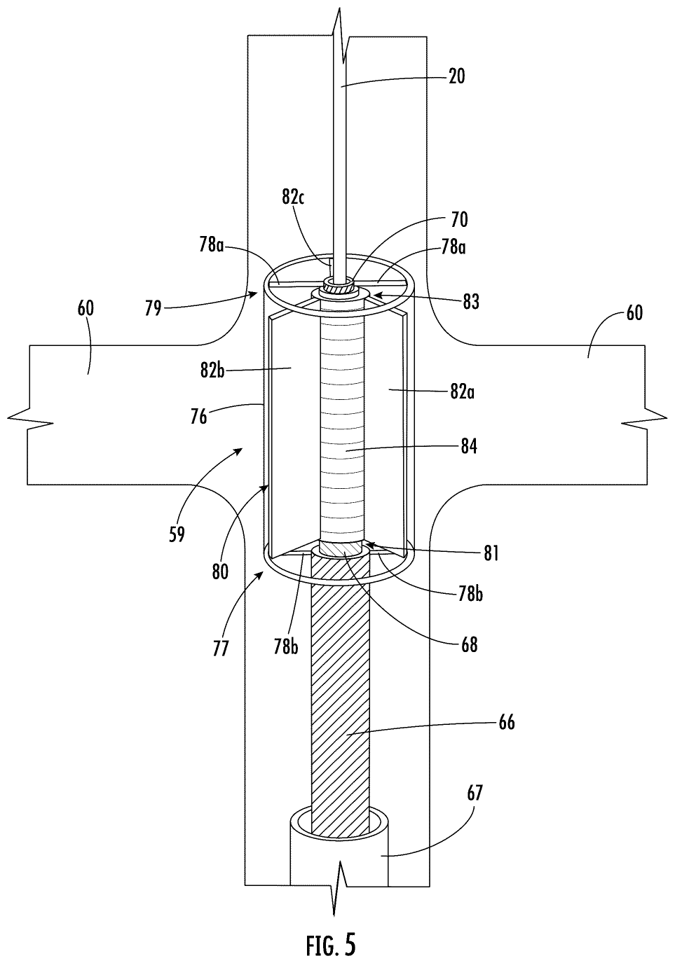

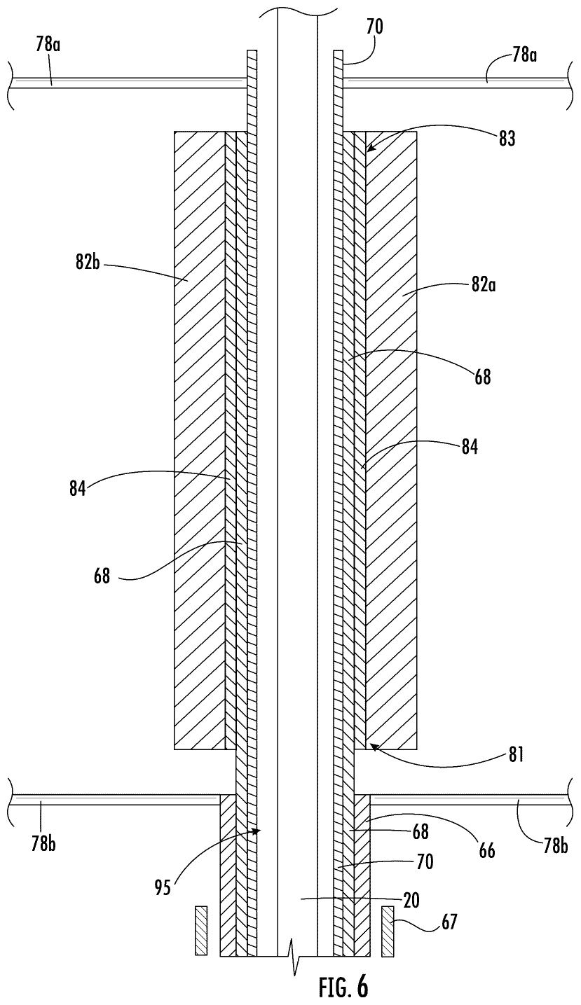

FIG. 4 illustrates that renal support device 54 may include an outer shaft 66 having a first end attached to the motor housing 74 and a second end coupled to the renal support pump 59 (discussed herein with respect to FIG. 5 and FIG. 6). FIG. 4 illustrates that the renal support system 54 may further include an inner catheter shaft 70 positioned radially inward of the outer shaft 66. The inner catheter shaft 70 may have a first end attached to a valve 23 and a second end coupled to the renal support pump 59 (discussed herein with respect to FIG. 5 and FIG. 6). The motor core 72 may be configured to rotate around the inner catheter shaft 70.

In some examples, a distal end region of the motor housing 74 may be coupled to an outer surface of the inner shaft 70. For example, in some examples, the motor housing 74 may extend circumferentially around the outer surface of the inner shaft 70.

FIG. 4 illustrates that the renal support device 54 may further include a co-axial electrical drive cable 68 positioned radially inward of the outer shaft 66 and radially outward of the inner catheter shaft 70 (e.g., co-axial electrical drive cable 68 may be sandwiched between the outer shaft 66 and the inner shaft 70). The electrical drive cable 68 may have a proximal end attached to the motor core 72. Accordingly, it can be appreciated that rotation of the motor core 72 may cause rotation of the electrical drive cable 68.

In the co-axial configuration, the electrical drive cable 68 may extend circumferentially around the inner surface of the outer shaft 66. As will be discussed in greater detail herein, the electrical drive cable 68 may be configured to rotate around the inner the shaft 70. Further, rotation of the electrical drive cable 68 around the inner shaft 70 may rotate an impeller assembly (shown in FIG. 5) of the renal pump device 59. The electrical drive cable 68 may rotate around the inner shaft 70 while the inner shaft remains stationary.

Additionally, FIG. 4 illustrates that the inner shaft 70 may include a lumen extending from the proximal end of the inner shaft 70 to the distal end of the inner shaft 70. It can be appreciated that both the valve 23 and the lumen of the inner shaft 70 may be sized to permit a medical device to extend therein. For example, FIG. 4 illustrates that the lumen of the inner catheter shaft 70 may be sized to permit the blood pump 24 and the catheter shaft 20 of the circulatory support device 12 to extend therein.

Further, FIG. 4 illustrates the proximal end of the catheter shaft 20 may include one or more features which permit the catheter shaft 20 to be disconnected from the handle 22. For example, the proximal end of the catheter shaft 20 may include a plug (e.g., a LEAD-style end plug) which is configured to disconnect from the handle 22. In some instances, the proximal end of the catheter shaft 20 may include a co-axial plug which is configured to mate with a co-axial receptacle positioned on the distal end region of the handle 22. The co-axial plug may be configured to disconnect from the co-axial receptacle positioned on the handle 22. However, it is contemplated that a variety of other connections may be utilized to connect the catheter shaft 20 to the handle 22.

FIG. 4 further illustrates that the delivery catheter 67 positioned radially outward of the outer shaft 66. The delivery catheter 67 may be configured to move relative to the outer shaft 66, the electrical drive cable 68, the inner shaft 70 and the renal support pump 59. For example, the outer shaft 67 may be configured to be advanced over the renal support pump 59, whereby the renal support pump 59 may collapse such that it can be positioned within the lumen of the delivery catheter 67 in a delivery configuration. Retraction of the delivery catheter 67 may permit the renal support pump to expand into a deployed configuration after being tracked to a target deployment site (e.g., the renal arteries).

FIG. 5 illustrates the renal support pump 59 positioned adjacent to the renal arteries 60 in a deployed configuration. The renal support pump 59 may include an impeller assembly 80 positioned within a frame 76. The impeller assembly 80 may include an impeller shaft 84. Referring to FIG. 6, which illustrates a partial cross-sectional view of the impeller assembly 80 shown in FIG. 5, the inner shaft 70 and the electrical drive cable 68 of the renal support system 54 may extend within the lumen of the impeller shaft 84. Further, FIG. 6 illustrates that the inner surface of the impeller shaft 84 may be fixedly attached to the electrical drive cable 68. As discussed herein, the electrical drive cable 68 may rotate relative to the inner shaft 70. Accordingly, rotation of the electrical drive cable 68 may rotate the impeller assembly 80 (via the attachment of the electrical drive cable 68 to the impeller shaft 84) around the inner shaft 70.

Referring to FIG. 5 and FIG. 6, the impeller assembly 80 may include a first impeller blade 82a, a second impeller blade 82b and a third impeller blade 82c (shown in FIG. 5, but not visible in FIG. 6). Each of the first impeller blade 82a, the second impeller blade 82b and the third impeller blade 82c may include a first longitudinal surface which is attached to the outer surface of the impeller shaft 84. Each of the first impeller blade 82a, the second impeller blade 82b and the third impeller blade 82c may extend from the proximal end 81 of the impeller shaft 84 to the distal end 83 of the impeller shaft 84. Additionally, in some examples, the first impeller blade 82a, the second impeller blade 82b and the third impeller blade 82c may be spaced circumferentially equidistant from one another around the longitudinal axis of the impeller shaft 84. In other examples, the first impeller blade 82a, the second impeller blade 82b and the third impeller blade 82c may not be spaced circumferentially equidistant from one another around the longitudinal axis of the impeller shaft 84. While FIG. 5 illustrates the impeller assembly 84 including three impeller blades 82a, 82b, 82c, it is contemplated that the impeller assembly 84 may include 1, 2,3, 4, 5, 6 or more impeller blades, whereby the impeller blades may be arranged in a variety of configurations around the impeller shaft 84.

FIG. 5 and FIG. 6 illustrate that the renal support pump 59 may further include a frame 76. The frame 76 may have a distal end region 79 and a proximal end region 77. FIG. 5 further illustrates that the impeller assembly 80 (including the first impeller blade 82a, the second impeller blade 82b and the third impeller blade 82c) may be positioned within the frame 76. Additionally, the distal end region 79 of the frame 76 may include one or more strut members 78a which extend radially inward and attach to the distal end region 83 of the inner shaft 70. Further, the proximal end region 77 of the frame 76 may include one or more strut members 78b which extend radially inward and attach to the proximal end region 81 of the outer shaft 66.

In some examples, the frame 76 may include an open-cell lattice structure extending from the proximal end region 77 to the distal end region 83 of the frame 76. The open-cell lattice structure may be formed from a plurality of interconnected strut members which define a plurality of open-cells extending therebetween. It can be appreciated that the open-cell lattice structure may permit blood to flow through the frame 76 in both a direction substantially parallel to the frame 76 and in a direction substantially perpendicular to the frame 76. Further, it can be appreciated that the frame 76 may be sized to permit the impeller assembly 80 to rotate while positioned within the frame 76. Accordingly, it can be appreciated that, when positioned adjacent the renal arteries, the frame 76 may be prevent the impeller assembly 80 from contacting the vessel walls adjacent the renal arteries 60 when the impeller assembly 80 is spinning within the frame 76. In other examples, the frame 76 may act to center the impeller assembly 80 within the body vessel adjacent to the renal arteries 60 when the impeller assembly 80 is spinning within the frame 76.

As discussed herein, it can be appreciated from FIG. 6 that the catheter shaft 20 of the circulatory support device 12 may extend within the lumen 95 of the inner shaft 70. Accordingly, it can be appreciated that the renal support pump 59 may be tracked over the catheter shaft 20 of the circulatory support device 12 when being positioned adjacent to the renal arteries 60.

FIG. 5 and FIG. 6 further illustrate the delivery catheter 67 positioned radially outward of the outer shaft 66. As discussed herein, the delivery catheter 67 may be configured to be advanced over the renal support pump 59, whereby the renal support pump 59 may collapse such that it can be positioned within the lumen of the delivery catheter 67 in a delivery configuration. Retraction of the delivery catheter 67 may permit the renal support pump to expand into a deployed configuration after being tracked to a target deployment site (e.g., the renal arteries).

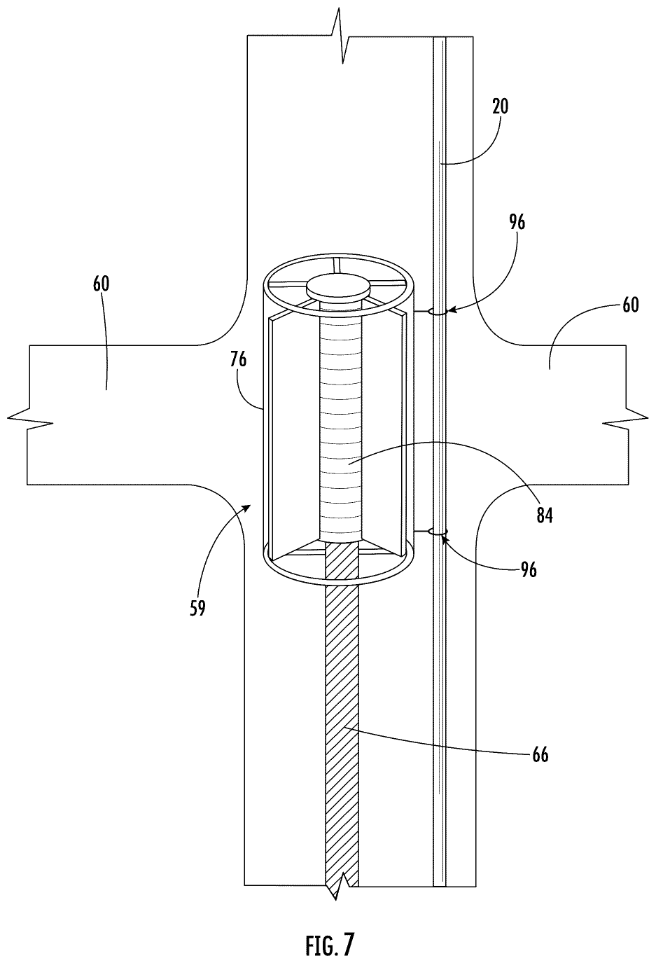

FIG. 7 illustrates another embodiment of the renal support pump 59. Specifically, FIG. 7 illustrates an embodiment in which the frame 76 of the renal support pump 59 includes one or more clips 96 designed to couple the renal support pump 59 to the catheter shaft 20 of the circulatory support device 12. It can be appreciated that the clips 96 may permit the renal support pump 59 to be tracked along the catheter shaft 20 of the circulatory support device 12 to a position adjacent the renal arteries 60. In this example, when positioned adjacent the renal arteries 60, the catheter shaft 20 and/or the renal support pump 59 may be offset from a central longitudinal axis of the body vessel adjacent the renal arteries 60. In other words, the catheter shaft 20 and/or the renal support pump 59 may be positioned adjacent to an inner surface of the body vessel adjacent the renal arteries 60.

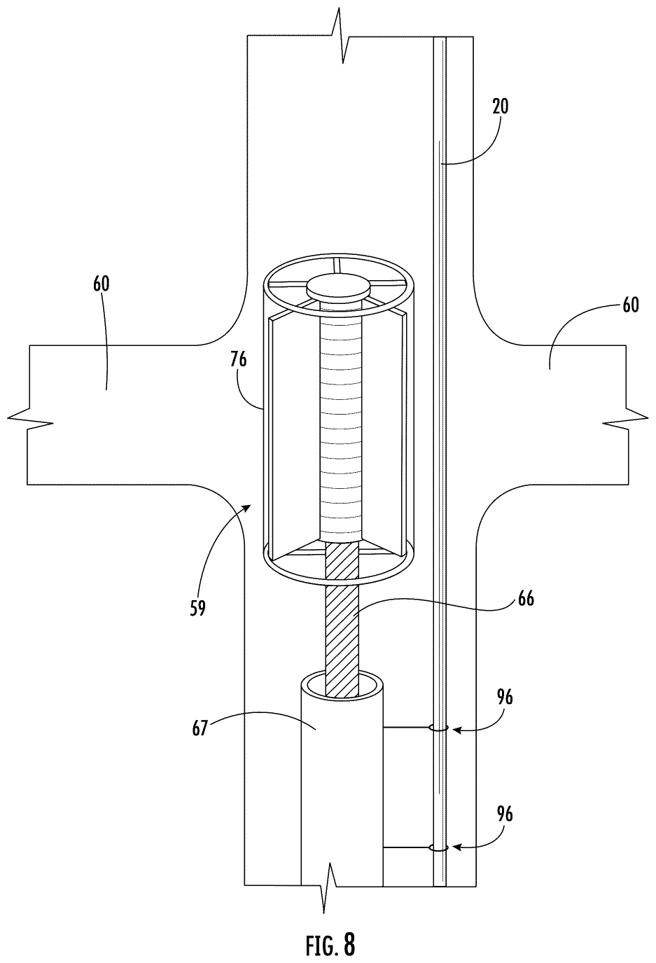

FIG. 8 illustrates another embodiment of the renal support pump 59. Specifically, FIG. 8 illustrates an embodiment in which the frame 76 of the renal support pump 59 includes one or more clips 96 designed to couple the delivery catheter 67 to the catheter shaft 20 of the circulatory support device 12. It can be appreciated that the clips 96 may permit the delivery catheter 67 to be tracked along the catheter shaft 20 of the circulatory support device 12 to a position adjacent the renal arteries 60. In this example, when positioned adjacent to the renal arteries 60, the catheter shaft 20, the renal support pump 59 and/or the delivery catheter 67 may be offset from a central longitudinal axis of the body vessel adjacent the renal arteries 60. In other words, the catheter shaft 20 and/or the renal support pump 59 may be positioned adjacent to an inner surface of the body vessel adjacent the renal arteries 60.

As discussed herein, the outer shaft 67 may be configured to be advanced over the renal support pump 59, whereby the renal support pump 59 may collapse such that it can be positioned within the lumen of the delivery catheter 67 in a delivery configuration. Retraction of the delivery catheter 67 may permit the renal support pump to expand into a deployed configuration after being tracked to a target deployment site (e.g., the renal arteries).

As discussed herein, patients who experience cardiogenic shock may need the support of a mechanical circulatory support system 10 to increase the blood flow and overall cardiac output of the heart. Additionally, acute kidney injury may be associated with cardiogenic shock. Therefore, the kidneys may be one of the first organs to be negatively impacted by cardiogenic shock. Further, increasing blood pressure beyond what is provided by the mechanical circulatory support system 10 may have the potential to increase renal function. Accordingly, placement and operation of the renal support pump 59 adjacent the renal arteries 60 may increase the pressure in the renal arteries 60 may permit the mechanical circulatory support system 10 to operate at a slightly lower pressure which may be beneficial in reducing hemolysis.

As discussed herein, the placement of the renal support pump 59 adjacent the renal arteries 60 may include tracking the renal support system 54 (including the renal support pump 59) over the catheter shaft 20 of the circulatory support device 12 which has been previously positioned in the patient 16. It can be appreciated that after the circulatory support device 12 has been positioned in the patient 16, the proximal end of the catheter shaft 20 may be disconnected from the handle 22. Accordingly, a clinician may then insert the proximal end of the catheter shaft 20 into the renal pump 59, whereby the renal support pump 59 may be tracked over the catheter shaft 20 to a position adjacent the renal arteries 60 (as shown in FIG. 1). After the renal support pump 59 is positioned adjacent to the renal arteries 60. After the renal support pump 59 is positioned adjacent the left ventricle 18, the inner catheter shaft 70 (which has passed through the valve 23 of the renal support device 54) may be reattached to the handle 22.

After the catheter shaft 20 is reattached to the handle 22, the clinician may activate (e.g., power up, turn on) the blood pump 24 via controls on the console 28a and/or the handle 22. Additionally, the clinician may activate (e.g., power up, turn on) the renal support pump 59 via controls on the console 28b. It can be appreciated that activation of the renal support pump 59 may include sending power from the console 28b to the motor core 72 of the motor assembly 62. Upon receiving power, the motor core 72 may begin to rotate relative to the inner shaft 70. Rotation of the motor core 72 may, in turn, rotate the electrical drive cable 68. Further, as discussed herein, rotation of the electrical drive cable 68 may cause the impeller assembly 80 (including the first impeller blade 82a, the second impeller blade 82b and the third impeller blade 82c) to rotate, whereby the rotation of the impeller assembly 80 may create a high-pressure wave at the entrance to the renal arteries 60, thereby providing support to the kidneys coincident with a circulatory support device being used to support the heart.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

1. A renal support device, comprising:
an outer catheter shaft having a longitudinal axis and a distal end region coupled to a renal pump assembly, wherein the renal pump assembly includes a frame and an impeller assembly, wherein the impeller assembly is disposed within the frame, and wherein the impeller assembly is configured to rotate relative to the frame about the longitudinal axis of the outer catheter shaft.

2. The renal support device of claim 1, wherein the impeller assembly includes a first impeller blade coupled to an impeller shaft.

3. The renal support device of claim 2, wherein the impeller shaft includes a first end region and a second end region, and wherein both the first end region and the second end region of the impeller shaft are coupled to an electrical drive cable.

4. The renal support device of claim 3, wherein the impeller shaft includes a lumen extending therein, and wherein the electrical drive cable is configured to extend within the lumen of the impeller shaft.

5. The renal support device of claim 4, wherein a proximal end of the electrical drive cable is coupled to a motor core.

6. The renal support device of claim 5, wherein the electrical drive cable and the motor core are configured to rotate around the impeller shaft.

7. The renal support device of claim 6, wherein the first impeller blade extends from the first end region of the impeller shaft to the second end region of the impeller shaft.

8. The renal support device of claim 1, further comprising a motor assembly including a motor core disposed within a motor housing, wherein the motor housing is coupled to the distal end region of the outer catheter shaft.

9. The renal support device of claim 8, wherein an electrical drive cable extends along an inner surface of the outer catheter shaft.

10. The renal support device of claim 9, wherein the electrical drive cable includes a lumen and wherein an inner catheter shaft is configured to extend within the lumen of the electrical drive cable.

11. The renal support device of claim 10, wherein the electrical drive cable extends circumferentially around an outer surface of the inner catheter shaft.

12. The renal support device of claim 10, wherein the motor core includes an aperture, and wherein the inner catheter shaft is configured to extend through the aperture of the motor core.

13. The renal support device of claim 8, further comprising a console coupled to the motor assembly.

14. The renal support device of claim 1, wherein the rotation of the impeller assembly is configured to create a high-pressure region within a body vessel of a patient.

15. A renal pump system, comprising:
a console including a processor;
a motor assembly coupled to the console; and
an outer catheter having a first end coupled to the motor assembly and a second end coupled to a renal pump;
wherein the renal pump includes a frame and an impeller assembly;
wherein the impeller assembly is configured to rotate relative to the frame, and wherein rotation of the impeller assembly is configured to create a high-pressure region within a body vessel of a patient.

16. The renal pump system of claim 15, wherein the impeller assembly includes a first impeller blade coupled to an impeller shaft.

17. The renal pump system of claim 16, wherein the impeller shaft includes a first end region and a second end region, and wherein both the first end region and the second end region of the impeller shaft are coupled to an electrical drive cable.

18. The renal pump system of claim 17, wherein the impeller shaft includes a lumen extending therein, and wherein the electrical drive cable is configured to extend within the lumen of the impeller shaft.

19. The renal pump system of claim 17, wherein the first impeller blade extends from the first end region of the impeller shaft to the second end region of the impeller shaft.

20. A method for positioning a renal pump system adjacent the renal arteries of a patient, the method comprising:
advancing a renal pump device adjacent to a renal artery of the patient, the renal pump device including:
an outer catheter shaft having a longitudinal axis and a distal end region coupled to the renal pump device, wherein the renal pump device includes a frame and an impeller assembly, wherein the impeller assembly is disposed within the frame, and wherein the impeller assembly is configured to rotate relative to the frame about the longitudinal axis of the outer catheter shaft.
